(19) United States
(12) Patent Application Publication
(10) Pub. No.: US 2026/0297097 A1
(43) Pub. Date: Oct. 1, 2026

(54) PROCESSES FOR PREPARING TRICYCLIC AMINES

(71) Applicant: Incyte Corporation, Wilmington, DE (US)

(72) Inventors: Jianguo Wu, Newtown, PA (US); Jiacheng Zhou, Newark, DE (US)

(21) Appl. No.: 19/572,073

(22) Filed: Mar. 19, 2026

Related U.S. Application Data
(60) Provisional application No. 63/774,968, filed on Mar. 20, 2025.

(51) Int. Cl. C07D 487/04

(52) U.S. Cl. CPC C07D 487/04

(57) ABSTRACT

This disclosure provides efficient and scalable processes for preparing the core structure of SPPL2a inhibitors.

### RELATED APPLICATIONS

This application claims priority to U.S. Provisional Application No. 63/774,968, filed Mar. 20, 2025, the content of which is incorporated in its entirety.

### BACKGROUND

Signal peptide peptidase-like 2a (SPPL2a) is an aspartic intramembrane protease that has been shown to play an important role in the development and function of antigen presenting cells such as B lymphocytes and dendritic cells (Beisner, D. R. et al., J. Exp. Med. 2013, 210, 23-30; Bergmann, H.; et al., J. Exp. Med. 2013, 210, 31-40; and Schneppenheim, J. et al., J. Exp. Med. 2013, 210, 41-58). Therefore, the inhibition of SPPL2a can reduce its antigen presenting capacity and could be explored as an approach for the treatment of autoimmune diseases (Hampe, C. S. Scientifica 2012, 2012, 215308; Mentrup, T. et al., Biochim. Biophys. Acta, Mol. Cell Res. 2017, 1864, 2169-2182).

Compounds having a tricyclic amino core have been shown to be potent SPPL2a inhibitors (WO 2022/058902). The preparation of this core structure, however, can be improved. The traditional preparation involves a two-step process starting from a tricyclic ketone that undergoes oxime formation followed by a reductive reaction (Velcicky, J., et al., J. Med. Chem. 2018. 61:865-880). The workup procedures and required chromatography in both the oxime formation and reductive reaction steps results in a relatively low yield (about 50% over two steps). As such, efficient and scalable synthetic routes are required to prepare SPPL2a inhibitors. The processes disclosed herein meet this need by providing a scalable synthetic route to prepare the tricyclic amino core of SPPL2a inhibitors.

### SUMMARY

Provided herein are processes for preparing a tricyclic amine that is Compound 1:

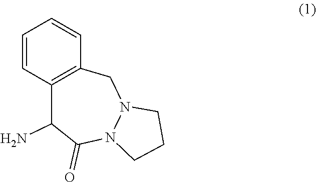 [SMILES: NC1C(=O)N2CCCN2Cc2ccccc21]

or a salt thereof;
   wherein the process comprises the step of reacting Compound 2:

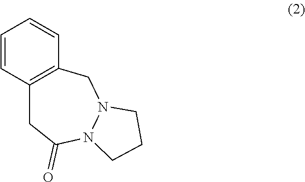 [SMILES: O=C1Cc2ccccc2CN2CCCN12]

or a salt thereof;
with an aminating agent and at least one base to produce Compound 1.

In an embodiment, this process for preparing Compound 1 is a one-step process.

Also provided herein are processes for preparing the tricyclic amine that is Compound 2:

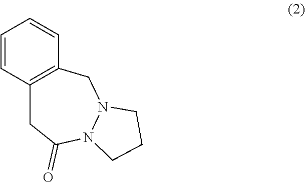 [SMILES: O=C1Cc2ccccc2CN2CCCN12]

wherein the process comprises the step of reacting Compound 3:

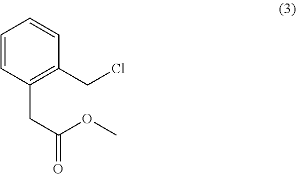

or a salt thereof;
   with Compound 4:

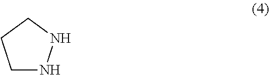

or a salt thereof;
in the presence of at least one base, a halogenating agent, and a solvent that is acetonitrile or an alcohol, to produce Compound 2.

In an embodiment, the processes described herein result in greater than 98% purity of the desired product. In an embodiment, the processes described herein result in greater than 80% yield of the desired product.

### DETAILED DESCRIPTION

Provided herein are efficient and scalable processes for preparing tricyclic amine compounds that are useful as SPPL2a inhibitors, or as intermediates of SPPL2a inhibitors. These inhibitors can be useful in the treatment of SPPL2a-mediated diseases, including autoimmune diseases and cancer.

### Definitions

Listed below are definitions of various terms used to describe the processes provided herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which the compound and its crystalline forms belong. Generally, the nomenclature used herein, and the laboratory procedures used in organic chemistry, and chemical manufacturing processes are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, “at least one” refers to a minimum of one and a maximum of five. For example, “at least one base” can refer to one base, two bases, three bases, four bases, or five bases.

The present disclosure also includes salts of the compounds described herein. The term “salt” refers to a derivative of the disclosed compounds wherein the parent compound is modified by converting an existing acid or base moiety to its salt form. Examples of salts include, but are not limited to, mineral or organic acid salts of basic residues such as amines; alkali or organic salts of acidic residues such as carboxylic acids; and the like. The salts of the present disclosure include the non-toxic salts of the parent compound formed, e.g., from non-toxic inorganic or organic acids. The salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, EtOAc, alcohols (e.g., MeOH, EtOH, iso-propanol or butanol) or MeCN are preferred. Lists of suitable salts are found in A. R. Gennaro (Ed.), Remington's Pharmaceutical Sciences, 17^{th} Ed., (Mack Publishing Company, Easton, 1985), p. 1418, S. M. Berge et al., J. Pharm. Sci., 1977, 66(1), 1-19, S. Gaisford in A. Adejare (Ed.), Remington, The Science and Practice of Pharmacy, 23^{rd} Ed., (Elsevier, 2020), Chapter 17, pp. 307-14; S. M. Berge et al., J. Pharm. Sci., 1977, 66(1), 1-19, T. S. Wiedmann, et al., Asian J. Pharm. Sci., 2016; 11, 722-34. D. Gupta et al., Molecules, 2018, 23(7), 1719; P. H. Stahl et al., Handbook of Pharmaceutical Salts: Properties, Selection, and Use, (Wiley, 2002) and in P. H. Stahl et al., Handbook of Pharmaceutical Salts: Properties, Selection, and Use, 2^{nd} Ed. (Wiley, 2011).

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±10%, including ±5%, ±1%, and ±0.1% from the specified value, as such variations are appropriate to perform the disclosed methods.

The expressions “ambient temperature” and “room temperature” are understood in the art, and refer generally to a temperature, e.g., a reaction temperature, which is about the temperature of the room in which the reaction is carried out, e.g., a temperature from about 20° C. to about 30° C.

As used herein, the term “reacting” refers to a chemical interaction between two chemical species that changes one set of chemical species into another. These interactions can be between a reactant and a reagent or a reactant and another reactant.

As used herein, the term “halogenating agent” refers to a reagent that installs a halo group as defined supra on a reactant. As such, a brominating agent installs a bromo group on a reactant and an iodinating agent installs an iodo group on a reactant. Examples of halogenating agents include, but are not limited to, elemental halogens, e.g., chorine, bromine, or iodine, interhalogen compounds, e.g., BrF₃, IF₅, ICl, and N-haloimides, e.g., NCS, NBS, or NIS.

As used herein, the phrase “aminating agent” refers to a chemical species that installs an amino group on a reactant by forming a carbon-nitrogen bond when combined with a nucleophile. Examples of aminating agents include, but are not limited to, O-diphenylphosphinyl hydroxylamine, monochloramine, O-(trimethylsilyl)hydroxylamine, hydroxyamine-O-sulfonic acid, O-tritylhydroxylamine, (2,4-dinitrophenyl)hydroxylamine, O-mesitylenesulfonylhydroxylamine, O-(2,4,6-triisopropylbenzenesulfonyl)hydroxylamine, O-(2,4,6-trinitrophenyl)hydroxylamine, O-(2,3,4,5,6-pentafluorobenzyl)hydroxylamine, O-(2,4,6-trimethylbenzoyl)hydroxylamine, O-(4 nitrobenzoyl)hydroxylamine, O-(2,4,6-trimethylbenzyl)hydroxylamine, and O-(4-nitrophenyl)hydroxylamine.

The reactions of the processes described herein can be carried out at appropriate temperatures that can be readily determined by the skilled artisan. Reaction temperatures will depend on, for example, the melting and boiling points of the reagents and solvent, if present; the thermodynamics of the reaction (e.g., vigorously exothermic reactions may need to be carried out at reduced temperatures); and the kinetics of the reaction (e.g., a high activation energy barrier may need elevated temperatures).

The reactions of the processes described herein can be carried out in air or under an inert atmosphere. Typically, reactions containing reagents or products that are substantially reactive with air can be carried out using air-sensitive synthetic techniques that are well known to the skilled artisan.

In some embodiments, preparation of compounds can involve the addition of acids or bases to effect, for example, catalysis of a desired reaction or formation of salt forms such as acid addition salts.

As used herein, the term “acid” refers to any species that can donate a proton or forming a covalent bond with an electron pair. Example acids can be inorganic or organic acids. Inorganic acids include hydrochloric acid (HCl), hydrobromic acid (HBr), sulfuric acid (H₂SO₄), phosphoric acid (H₃PO₄), perchloric acid (HClO₄), and nitric acid (HNO₃). Organic acids include formic acid, acetic acid, propionic acid, butanoic acid, benzoic acid, 4-nitrobenzoic acid, methanesulfonic acid, p-toluenesulfonic acid, benzenesulfonic acid, tartaric acid, trifluoroacetic acid (CF₃CO₂H), propiolic acid, butyric acid, 2-butynoic acid, vinyl acetic acid, pentanoic acid, hexanoic acid, heptanoic acid, octanoic acid, nonanoic acid and decanoic acid.

As used herein, the term “base” refers to any species that contains a filled orbital containing an electron pair which is not involved in bonding. Example bases include lithium hydroxide (LiOH), sodium hydroxide (NaOH), potassium hydroxide (KOH), lithium carbonate (Li₂CO₃), sodium carbonate (Na₂CO₃), potassium carbonate (K₂CO₃), cesium carbonate (Cs₂CO₃), potassium tert-butoxide (KOtBu), sodium tert-butoxide (NaOtBu), sodium ethoxide (NaOEt), sodium methoxide (NaOMe), and potassium methoxide (KOMe). Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include sodium and potassium salts of methyl, ethyl, n-propyl, i-propyl, n-butyl, t-butyl, TMS and cyclohexyl substituted amides. Examples of bases also include alkali metal acetate salts, such as sodium acetate or potassium acetate. Bases used herein can be a non-nucleophilic base, which is a base that is poor at sharing electrons due to steric hindrance. Examples of non-nucleophilic bases include potassium bis(trimethylsilyl)amide (KHMDS), sodium bis(trimethylsilyl)amide (NaHMDS), lithium bis(trimethylsilyl)amide (LiHMDS), lithium diisopropylamide (LDA), sodium hydride (NaH), potassium hydride (KH), 1,8-diazabicyclo[5.4.0]undec-7-ene (DBU), and N,N-diisopropylethylamine (DIPEA).

### Processes

### Preparation of Compound 1

In an aspect, provided herein is a process for preparing Compound 1:

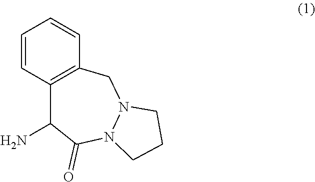 [SMILES: NC1C(=O)N2CCCN2Cc2ccccc21]

or a salt thereof;
   wherein the process comprises the step of reacting Compound 2:

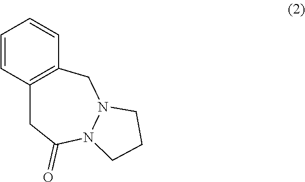 [SMILES: O=C1Cc2ccccc2CN2CCCN12]

or a salt thereof;
with an aminating agent and at least one base to produce Compound 1.

In an embodiment, the process is a one-step process.

In another embodiment, Compound 2 is reacted with one base. In another embodiment, Compound 2 is reacted with two bases.

In an embodiment, the base is a non-nucleophilic base. In another embodiment, the base is selected from potassium bis(trimethylsilyl)amide (KHMDS), sodium bis(trimethylsilyl)amide (NaHMDS), lithium bis(trimethylsilyl)amide (LiHMDS), lithium diisopropylamide (LDA), sodium hydride (NaH), potassium hydride (KH), 1,8-diazabicyclo[5.4.0]undec-7-ene (DBU), and N,N-diisopropylethylamine (DIPEA). In another embodiment, the base is potassium bis(trimethylsilyl)amide.

In an embodiment, the base is a metal alkoxide. In another embodiment, the base is selected from potassium tert-butoxide (KOtBu), sodium tert-butoxide (NaOtBu), sodium ethoxide (NaOEt), sodium methoxide (NaOMe), and potassium methoxide (KOMe). In another embodiment, the base is potassium tert-butoxide.

In an embodiment, Compound 2 is reacted with two bases wherein one base is potassium bis(trimethylsilyl)amide and another base is potassium tert-butoxide. In an embodiment, the base is potassium bis(trimethylsilyl)amide or potassium tert-butoxide.

In some embodiments, the process occurs in the presence of an excess of base. In some embodiments, the process occurs in the presence of about 1 to about 2 equivalents of base compared to Compound 2. In some embodiments, the process occurs in the presence of about 1 equivalent of base. In some embodiments, the process occurs in the presence of about 1.2 equivalents of base. In some embodiments, the process occurs in the presence of about 1.4 equivalents of base. In some embodiments, the process occurs in the presence about of 1.6 equivalents of base. In some embodiments, the process occurs in the presence of about 1.8 equivalents of base. In some embodiments, the process occurs in the presence of about 2 equivalents of base.

In an embodiment, the aminating agent is selected from O-diphenylphosphinyl hydroxylamine, monochloramine, O-(trimethylsilyl)hydroxylamine, hydroxyamine-O-sulfonic acid, O-tritylhydroxylamine, (2,4-dinitrophenyl)hydroxylamine, O-mesitylenesulfonylhydroxylamine, O-(2,4,6-triisopropylbenzenesulfonyl)hydroxylamine, O-(2,4,6-trinitrophenyl)hydroxylamine, O-(2,3,4,5,6-pentafluorobenzyl)hydroxylamine, O-(2,4,6-trimethylbenzoyl)hydroxylamine, O-(4 nitrobenzoyl)hydroxylamine, O-(2,4,6-trimethylbenzyl)hydroxylamine, and O-(4-nitrophenyl)hydroxylamine. In another embodiment, the aminating agent is O-diphenylphosphinyl hydroxylamine.

In some embodiments, the process occurs in the presence of an excess of aminating agent. In some embodiments, the process occurs in the presence of about 1 to about 2 equivalents of aminating agent compared to Compound 2. In some embodiments, the process occurs in the presence of about 1 equivalent of aminating agent. In some embodiments, the process occurs in the presence of about 1.2 equivalents of aminating agent. In some embodiments, the process occurs in the presence of about 1.4 equivalents of aminating agent. In some embodiments, the process occurs in the presence about of 1.6 equivalents of aminating agent. In some embodiments, the process occurs in the presence of about 1.8 equivalents of aminating agent. In some embodiments, the process occurs in the presence of about 2 equivalents of aminating agent.

In an embodiment, the process occurs in the presence of a solvent. In another embodiment, the solvent is a polar, aprotic solvent. In some embodiment, the solvent is selected from tetrahydrofuran, ethyl acetate, acetone, acetonitrile, dichloromethane, pyridine, and dimethylformamide. In an embodiment, the solvent is tetrahydrofuran.

In some embodiments, the reaction can be carried out at a temperature in the range from about 0° C. to about −100° C., such as a temperature of about −20° C. to about −80° C., about −20° C. to about −60° C., about −20° C. to about −40° C., and about −25° C. to about −35° C. In some embodiments, the reaction can be carried out at a temperature in the range from about −25° C. to about −35° C. In some embodiments, the reaction can be carried out at a temperature of about −60° C. In some embodiments, the reaction can be carried out at a temperature of about −10° C.

In an embodiment, the process comprises preparing Compound 1, or a salt thereof, comprising the step of reacting Compound 2, or a salt thereof, with O-diphenylphosphinyl hydroxylamine and potassium bis(trimethylsilyl)amide. In an embodiment, the process occurs in the presence of THF.

In an embodiment, the process comprises preparing Compound 1, or a salt thereof, comprising the step of reacting Compound 2, or a salt thereof, with O-diphenylphosphinyl hydroxylamine and potassium tert-butoxide. In an embodiment, the process occurs in the presence of THF.

In an embodiment, the process comprises preparing Compound 1, or a salt thereof, comprising the step of reacting Compound 2, or a salt thereof, with O-diphenylphosphinyl hydroxylamine, potassium bis(trimethylsilyl)amide, and potassium tert-butoxide. In an embodiment, the process occurs in the presence of THF.

In an embodiment, the process comprises an acid-base extraction.

In an embodiment, the acid-base extraction comprises the following step of treating Compound 1 with an acid to generate a salt of Compound 1.

In an embodiment, the acid is a strong acid. In an embodiment, the acid is selected from hydrochloric acid (HCl), nitric acid (HNO₃), sulfuric acid (H₂SO₄), hydrobromic acid (HBr), perchloric acid (HClO₄), and trifluoroacetic acid (CF₃CO₂H). In an embodiment, the acid is hydrochloric acid.

In an embodiment, the salt of Compound 1 is washed with a polar, aprotic solvent. In an embodiment, the solvent is selected from dichloromethane, chloroform, tetrahydrofuran, ethyl acetate, acetonitrile, dimethylformamide, N-methylpyrrolidone, and pyridine. In an embodiment, the solvent is selected from dichloromethane, tetrahydrofuran, ethyl acetate, acetonitrile, and pyridine. In an embodiment, the solvent is dichloromethane.

In an embodiment, the acid-base extraction comprises the following step of treating the salt of Compound 1 with a base to generate Compound 1 as a free base.

In an embodiment, the base is a strong base. In an embodiment, the base is selected from sodium hydroxide (NaOH), potassium hydroxide (KOH), lithium hydroxide (LiOH), potassium tert-butoxide (KOtBu), sodium tert-butoxide (NaOtBu), sodium ethoxide (NaOEt), sodium methoxide (NaOMe), and potassium methoxide (KOMe). In an embodiment, the base is sodium hydroxide.

In an embodiment, the Compound 1 is washed with a polar, aprotic solvent. In an embodiment, the solvent is selected from dichloromethane, chloroform, tetrahydrofuran, ethyl acetate, acetonitrile, dimethylformamide, N-methylpyrrolidone, and pyridine. In an embodiment, the solvent is selected from dichloromethane, tetrahydrofuran, ethyl acetate, acetonitrile, and pyridine. In an embodiment, the solvent is dichloromethane.

In an embodiment, the acid-base extraction comprises
a) treating Compound 1 with an acid to generate a salt of Compound 1;
b) washing the salt of Compound 1 with a polar, aprotic solvent;
c) treating the salt of Compound 1 with a base to generate Compound 1 as a free base; and
d) extracting Compound 1 free base with a polar, aprotic solvent.

In another embodiment, the acid-base extraction comprises
a) treating Compound 1 with hydrochloric acid to generate a salt of Compound 1;
b) washing the salt of Compound 1 with dichloromethane;
c) treating the salt of Compound 1 with sodium hydroxide to generate Compound 1 as a free base; and
d) extracting Compound 1 free base with dichloromethane.

In an embodiment, Compound 1 is prepared at an overall yield of about 80% or greater. In an embodiment, Compound 1 is prepared at an overall yield of about 75% to about 90%. In an embodiment, Compound 1 is prepared at an overall yield of about 80% to about 85%.

In another embodiment, Compound 1 is prepared at an overall purity of greater than 99%. In another embodiment, Compound 1 is prepared at an overall purity of about 99%.

In an embodiment, Compound 1 is prepared without a chromatography step. In an embodiment, Compound 1 is prepared without chromatography purification.

### Preparation of Compound 2

In an aspect, provided herein is a process for preparing Compound 2:

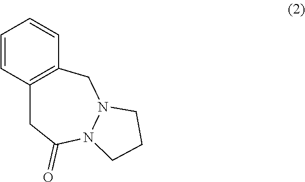 [SMILES: O=C1Cc2ccccc2CN2CCCN12]

wherein the process comprises the step of reacting Compound 3:

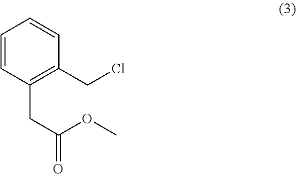

or a salt thereof;
   with Compound 4:

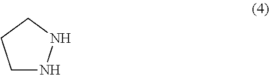

or a salt thereof;

in the presence of at least one base, a halogenating agent, and a solvent that is acetonitrile or an alcohol, to produce Compound 2.

In an embodiment, the solvent is acetonitrile. In an embodiment, the solvent is an alcohol. In another embodiment, the solvent is ethanol or methanol. In another embodiment, the solvent is ethanol. In another embodiment, the solvent is methanol.

In an embodiment, the process occurs in the presence of one base. In an embodiment, the process occurs in the presence of two bases.

In an embodiment, the base is a non-nucleophilic base. In another embodiment, the base is selected from potassium bis(trimethylsilyl)amide (KHMDS), sodium bis(trimethylsilyl)amide (NaHMDS), lithium bis(trimethylsilyl)amide (LiHMDS), lithium diisopropylamide (LDA), sodium hydride (NaH), potassium hydride (KH), 1,8-diazabicyclo[5.4.0]undec-7-ene (DBU), and N,N-diisopropylethylamine (DIPEA). In another embodiment, the base is N,N-diisopropylethylamine.

In an embodiment, the base is an alkali metal acetate salt. In another embodiment, the base is sodium acetate or potassium acetate. In another embodiment, the base is sodium acetate.

In an embodiment, the halogenating agent is an iodinating agent. In another embodiment, the halogenating agent is selected from sodium iodide, N-iodosuccinimide, iodine chloride, 1,3-diiodo-5,5-dimethylhydantoin, N-iodosaccharin, and bis(pyridine)iodonium tetrafluoroborate. In another embodiment, the halogenating agent is sodium iodide.

In an embodiment, the process comprises preparing Compound 2 by reacting Compound 3, or a salt thereof, with Compound 4, or a salt thereof, in the presence of N,N-diisopropylethylamine, sodium acetate, and sodium iodide in a solvent that is acetonitrile.

In an embodiment, the process comprises preparing Compound 2 by reacting Compound 3, or a salt thereof, with Compound 4, or a salt thereof, in the presence of N,N-diisopropylethylamine, sodium acetate, and sodium iodide in a solvent that is ethanol.

In an embodiment, the process occurs at room temperature. In some embodiments, the process occurs at room temperature for about 1 hour to about 10 hours. In some embodiments, the process occurs at room temperature for about 2 hours to about 8 hours. In some embodiments, the process occurs at room temperature for about 4 hours to about 6 hours. In some embodiments, the process occurs at room temperature for about 5 hours.

In another embodiment, the reaction is heated after reacting at room temperature.

In some embodiments, the reaction is heated to about 50° C. to about 150° C., such as about 60° C. to about 120° C., about 70° C. to about 100° C., about 70° C. to about 90° C., or about 70° C. to about 80° C., after reacting at room temperature.

In another embodiment, the reaction is heated to about 70° C. to about 80° C. after reacting at room temperature. In another embodiment, the reaction is heated to about 75° C. to about 80° C. after reacting at room temperature.

In some embodiments, the reaction is heated for about 1 hour to about 10 hours. In some embodiments, the reaction is heated for about 2 hours to about 8 hours. In some embodiments, the reaction is heated for about 4 hours to about 6 hours. In some embodiments, the reaction is heated for about 5 hours. In some embodiments, the reaction is heated for about 6 hours.

In an embodiment, Compound 2 is prepared at an overall yield of about 85% or greater. In an embodiment, Compound 1 is prepared at an overall yield of about 85% to about 95%. In an embodiment, Compound 1 is prepared at an overall yield of about 85% to about 90%.

In another embodiment, Compound 2 is prepared at an overall purity of greater than 98%. In another embodiment, Compound 2 is prepared at an overall purity of about 97% to about 99%. In another embodiment, Compound 2 is prepared at an overall purity of about 98% to about 99%. In another embodiment, Compound 2 is prepared at an overall purity of about 98%.

In an embodiment, the ratio of Compound 2 produced to byproduct produced is about 4:1. In another embodiment, the ratio of Compound 2 produced to byproduct produced is about 4.5:1. In another embodiment, the ratio of Compound 2 produced to byproduct produced is about 5:1. In an embodiment, the ratio of Compound 2 produced to byproduct produced is a molar ratio.

In another embodiment, the byproduct is Compound 5:

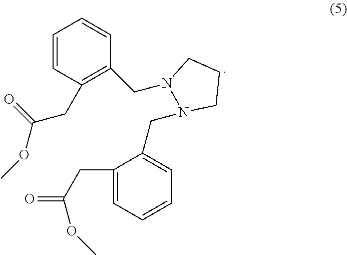 [SMILES: COC(=O)Cc1ccccc1CN1CCCN1Cc1ccccc1CC(=O)OC]

In some embodiments, the process occurs in the presence of about 1 equivalent of Compound 4 compared to Compound 3. In some embodiments, the process occurs in the presence of about 1 equivalent to about 2 equivalents of Compound 4. In some embodiments, the process occurs in the presence of about 1.2 equivalent to about 1.8 equivalents of Compound 4. In some embodiments, the process occurs in the presence of about 1.4 equivalent to about 1.6 equivalents of Compound 4. In some embodiments, the process occurs in the presence of about 1.5 equivalents of Compound 4. In some embodiments, the process occurs in the presence of about 2 equivalents of Compound 4.

In some embodiments, the process occurs in the presence of an excess of base. In some embodiments, the process occurs in the presence of about 4 equivalents to about 5 equivalents of base compared to Compound 3. In some embodiments, the process occurs in the presence of about 5 equivalents of base. In some embodiments, the process occurs in the presence of about 4 equivalents of base. In some embodiments, the process occurs in the presence of about 5 equivalents of one base and about 4 equivalents of a second base.

In some embodiments, the process occurs in the presence of an excess of halogenating agent. In some embodiments, the process occurs in the presence of about 1 equivalent to about 2 equivalents of halogenating agent compared to Compound 3. In some embodiments, the process occurs in the presence of about 1 equivalent to about 1.5 equivalents of halogenating agent. In some embodiments, the process occurs in the presence of about 1 equivalent to about 1.2 equivalents of halogenating agent. In some embodiments, the process occurs in the presence of about 1.1 equivalents of halogenating agent. In some embodiments, the process occurs in the presence of about 1.2 equivalents of halogenating agent.

In an embodiment, provided herein is a process for preparing Compound 1, or a salt thereof, comprising the step of reacting Compound 3, or a salt thereof, with Compound 4, or a salt thereof, in the presence of at least one base, a halogenating agent, and a solvent that is acetonitrile or an alcohol, to produce Compound 2; and the step of reacting Compound 2 with an aminating agent and at least one base to produce Compound 1.

Those skilled in the art will recognize or be able to ascertain using no more than routine experimentation, numerous equivalents to the specific procedures, embodiments, claims, and examples described herein. Such equivalents are within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### Examples

The disclosure is further illustrated by the following examples, which should not be construed as further limiting. The practice of the present disclosure will employ, unless otherwise indicated, conventional techniques of organic synthesis, which are within the skill of the art.

### Example 1: Optimization of the Preparation of Compound 1

Direct electrophilic amination, as described in the present disclosure, has significant advantages, such as improved yield, purity, reaction time, reaction steps, safety, etc., over classical amination methods that involve the reaction of a nitrogen nucleophile with an electrophilic carbon center. One classical amination method involves the use of monochloramine (NH₂Cl). Monochloramine is one of the cheapest and most simple electrophilic aminating reagents. It is prepared as a solution in Et₂O by a reaction of NH₄Cl, NH₄OH and NaOCl (in the form of commercial bleach). As shown in Scheme 1, deprotonation of tricyclic ketone 2 in the a position with LiHMDS at −30° C. followed by 1.4 eq NH₂Cl ethereal solution produced tricyclic amine 1 with 80% conversion and 56% assay yield. Modifications to the reaction conditions including extra LiHMDS and/or NH₂Cl, and variation of the reaction time and temperature did not increase the conversion. Further, the instability and toxicity of monochloramine limits its large-scale use.

Other electrophilic aminations of 2 with various aminating reagents were evaluated with the results summarized in Table 1.

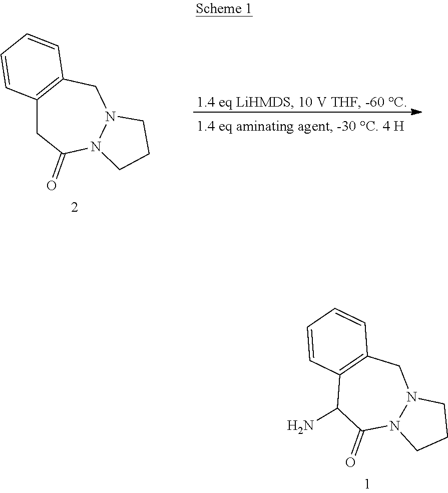 [SMILES: O=C1Cc2ccccc2CN2CCCN12 ; NC1C(=O)N2CCCN2Cc2ccccc21]

TABLE 1

| Entry | Aminating reagents | Conversion (%) |
|---|---|---|
| 1 | NH₂Cl | 80 |
| 2 | Me₃SIONH₂ | 0 |
| 3 | Ph₃CONH₂ | 0 |
| 4 | DNPH | 39 |
| 5 | HOSA | 50 |
| 6 | MSH | 80 |
| 7 | DPPH | 82 |

The frequently applied commercially available hydroxyamine-O-sulfonic acid (HOSA) gave a moderate conversion. Other commercially available reagents like Me₃Si—O—NH₂ and Ph₃C—O—NH₂ (entries 2 and 3) were not effective. (2,4-Dinitrophenyl)hydroxylamine (DNPH) gave a low conversion (entry 4). Among all of the tested reagents with a hydroxyamine moiety, O-mesitylenesulfonylhydroxylamine (MSH) and O-(diphenylphosphinyl)hydroxylamine (DPPH) demonstrated the best performance giving high conversions (80%). However, reports of explosive decomposition limit the commercial availability of MSH and limit scalability of reactions.

In contrast, DPPH is commercially available, has much higher stability, and can be stored for long periods at room temperature. DPPH can also withstand relatively harsh conditions before it degrades. The most substantive benefit observed in the initial reactions tested is that the reaction of electrophilic amination of 2 with DDPH is clean and did not produce any major observable side products. Based on these observed advantages, DDPH was further tested as the aminating reagent for the preparation of tricyclic amine 1.

Using DPPH as the reagent for amination, reaction conditions were varied to improve the yield. Bases, stoichiometry, and reaction temperature were explored in detail and the results are summarized in Table 2.

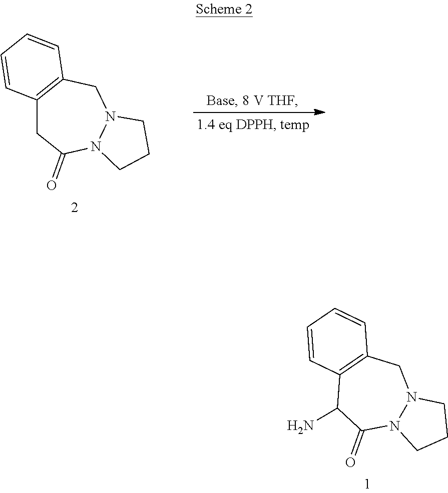 [SMILES: O=C1Cc2ccccc2CN2CCCN12 ; NC1C(=O)N2CCCN2Cc2ccccc21]

TABLE 2

| | Base | DPPH | Temp | Time | Conversion |
|---|---|---|---|---|---|
| Entry | (eq) | (eq) | (° C.) | (h) | (%) |
| 1 | LiHMDS (1.4) | 1.4 | −25 to −35 | 3 | 82 |
| 2 | NaHMDS (1.4) | 1.4 | −25 to- −35 | 3 | 89 |
| 3 | KHMDS (1.4) | 1.4 | −25 to −35 | 3 | 98.5 |
| 4 | KOtBu (1.4) | 1.4 | −25 to −35 | 3 | 98.7 |
| 5 | KHMDS (1.4) | 1.4 | −60 | 3 | 56 |
| 6 | KHMDS (1.4) | 1.4 | −60 | 9 | 56 |
| 7 | KHMDS (1.4) | 1.4 | −10 | 9 | 52 |
| 8 | KHMDS (1.2) | 1.2 | −25 to −35 | 3 | 85 |
| 9 | KHMDS (1.6) | 1.6 | −25 to −35 | 3 | 98.5 |

No desired amination product was observed when BuLi was used as a base, but LiHMDS can provide 82% conversion. Increased conversion was observed for the sodium and potassium bases shown in Table 2. Both KHMDS and KO^{t}Bu provided excellent conversion (>98%) at around −30° C. (entries 3, 4), but did not react completely at excessively low temperatures (entries 5, 6) with 56% conversion. Interestingly, a similar low conversion was observed at high reaction temperatures (entry 7), which could be due to the instability of potassium enolate which decomposes at −10° C. The best reaction temperature was observed to be between −20° C. and −35° C.

The quality and amount of base and DPPH also affected the conversion. Less base and DPPH reduced the conversion (entry 8) but too much base and DPPH was not required for high conversion (entry 9). 1.4 Equivalents of a potassium base (KHMDS or KO^{t}Bu) and 1.4 equivalents DPPH were found to be sufficient for exceptional conversion.

After the amination was completed, water was added to quench the reaction. After adding excess methyl tert-butyl ether (MTBE), most of the DPPH byproduct, Ph₂P(O)OK, crashed out of the THF/water solution as a solid and was removed by filtration. Evaporation of the filtrate gave ~70% pure Compound 1 and ~30% of PH₂P(O)OK and other minor byproducts related to DPPH.

Addition of excess hexanes to THF/water solution not only crashed out the Ph₂P(O)OK byproduct but also 2% of desired Compound 1. To reduce the loss of the product, a more polar solvent, MTBE, was tested. When using MTBE less than 0.6% of desired Compound 1 crashed out with the Ph₂P(O)OK byproduct.

Unfortunately, further purification proved extremely difficult. Compound 1 has some solubility both in water and the organic solvent THF. Thus, in an effort to purify Compound 1, the aqueous filtrate was treated with 3 N HCl until pH 2 to generate the HCl salt of Compound 1. Washing this acidic aqueous solution with dichloromethane (DCM) effectively removed the impurities and left pure HCl salt of Compound 1 in aqueous solution.

The free base of Compound 1 was regenerated by saponification of the resulting acidic solution with 5 N NaOH. Further extraction by DCM, evaporation, and drying afforded Compound 1. This acid-base extraction proved extremely effective as Compound 1 was obtained with excellent purity (>98%) and no chromatography needed.

As such, the Compound 1 can be prepared at high yield and excellent purity by the following procedures.

### From 1.0M KHMDS in THF

To a suspension of DPPH (39.8 g, 171 mmol, Combi-Block) and 2 (23 g, 114 mmol) in THF (280 mL, 3412 mmol) at −35° C. was added 1.0 M KHMDS in THF (159 mL, 159 mmol). The mixture was stirred at −25° C. to −35° C. for 3 h. After warming to 0° C., the reaction mixture was quenched with water (15 mL), followed by the addition of MTBE (460 mL). The mixture was filtered, and the filtrate was adjusted to pH 2 by 2N HCl (138 mL). After the organic solvents were evaporated, the remaining aqueous solution was washed with DCM (230 mL) and then adjusted to pH 12 by 5N NaOH (69 mL). The basic aqueous solution was extracted with DCM (2×230 ml). The combined organic extractions were dried over Na₂SO₄, concentrated and dried to provide the desired product (20.1 g, 81% yield) as a red thick oil. LC-MS calculated for C₁₂H₁₅N₃O (M+H)⁺: m/z=218.1; found 218.1. ¹H NMR (400 MHz, DMSO-d₆) δ ppm 7.72-7.75 (m, 1H), 7.16-7.22 (m, 2H), 7.00-7.03 (m, 1H), 5.57 (s, 1H), 4.21 (s, 2H), 3.49-3.56 (m, 2H), 3.13-3.28 (m, 2H), 3.01 (br s, 2H), 2.28-2.40 (m, 1H), 2.05-2.14 (m, 1H).

From solid potassium tert-butoxide (KO^{t}Bu)

To a suspension of DPPH (363 g, 1.557 mol) and 2 (210 g, 1.048 mol) in THF (4.1 L) at −35° C. was added potassium tert-butoxide (163 g, 1.45 mol) in several portions. The mixture was stirred at −25° C. to −35° C. for 3 h. After warming to 0° C., the reaction mixture was quenched with water (126 mL), followed by the addition of tert-butyl methyl ether (4.2 L). The mixture was filtered, and the filtrate was adjusted to pH 2 by 3N HCl (0.42 L). After the organic solvents were evaporated, the remained aqueous solution was washed with CH₂Cl₂ (2×1 L) and then adjusted to pH 12 by 5N NaOH (0.23 L). The basic aqueous solution was extracted with CH₂Cl₂ (2×1.6 L). The combined organic extractions were dried over Na₂SO₄, concentrated and dried to provide the desired product (185 g, 86% yield) as a red thick oil.

### Example 2: Optimization of the Preparation of Compound 2

The published synthesis of tricyclic ketone 2 is shown in Scheme 3 (WO 2022/058902).

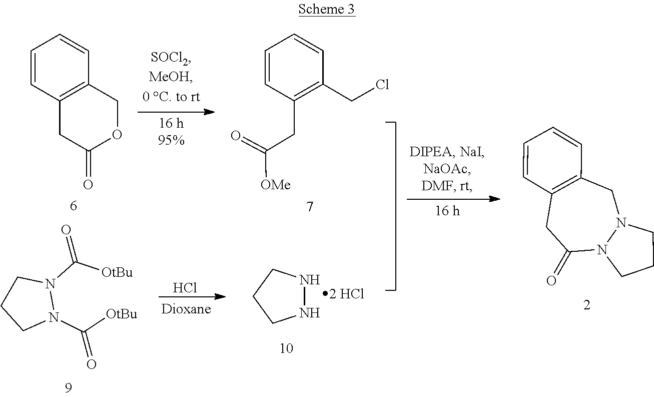 [SMILES: CC(C)(C)OC(=O)N1CCCN1C(=O)OC(C)(C)C ; O=C1Cc2ccccc2CO1 ; Cl ; COC(=O)Cc1ccccc1CCl ; C1CNNC1 ; O=C1Cc2ccccc2CN2CCCN12]

The chloroester 7 was prepared by a thionyl chloride mediated ring opening of commercially available isochromanone 6 while pyrazolidine 10 was prepared from deprotecting compound 9 which was prepared following a literature method (Shen, Y., et al., J. Org. Chem. 2002, 67, 6236-6239). The cyclization of pyrazolidine 10 with chloroester 7 in DMF at room temperature for 16 h in the presence of DIPEA, NaI, and NaOAc produced Compound 2 with 57% isolated yield. While attempting to replicate the preparation of Compound 2, achieving similar yields proved to be a significant challenge. Following the same procedure at room temperature for 16 h, only 10-15% of Compound 2 was produced. High reaction temperature (100° C., 16 h) modestly increased the yields to 40-50%. After most DMF solvent was removed, analysis of the crude residue by LC-MS revealed a major byproduct: Compound 11 in Scheme 4. In addition to low conversion, removing DMF either by evaporation and/or extraction during workup proved tedious. To overcome these issues, an improved method was optimized to increase the yields of Compound 2 to about 80% with a much simpler workup procedure.

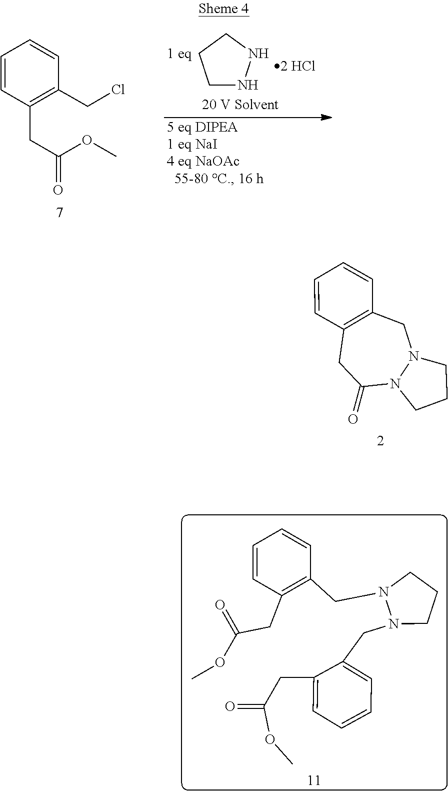 [SMILES: COC(=O)Cc1ccccc1CCl ; C1CNNC1 ; O=C1Cc2ccccc2CN2CCCN12 ; COC(=O)Cc1ccccc1CN1CCCN1Cc1ccccc1CC(=O)OC]

As described above, elevating the reaction temperature increased the yield of tricyclic ketone 2. To find alternative low boiling solvents to replace DMF, several solvents were screened for the cyclization reaction at 80° C. (Table 3).

TABLE 3

| | | | HPLC area percent (%) | | |
|---|---|---|---|---|---|
| Entry | Solvents | Temp (° C.) | 2 | 11 | 7 |
| 1 | CH₃CN | 80 | 75.4 | 16.0 | 0 |
| 2 | EtOH | 78 | 68.6 | 14.3 | 0 |
| 3 | THF | 74 | 30.9 | 25.6 | 12.4 |
| 4 | Acetone | 55 | 2.5 | 2.5 | 7.9 |

Acetonitrile (CH₃CN) provided the best results with 75.4% yield of Compound 2 (Table 1, entry 1), however, a high percent (16%) of byproduct 11 caused the low yield. To pinpoint when byproduct 11 is produced, the reaction was monitored at 80° C. from 0 h to 6 h (Scheme 5, Table 4).

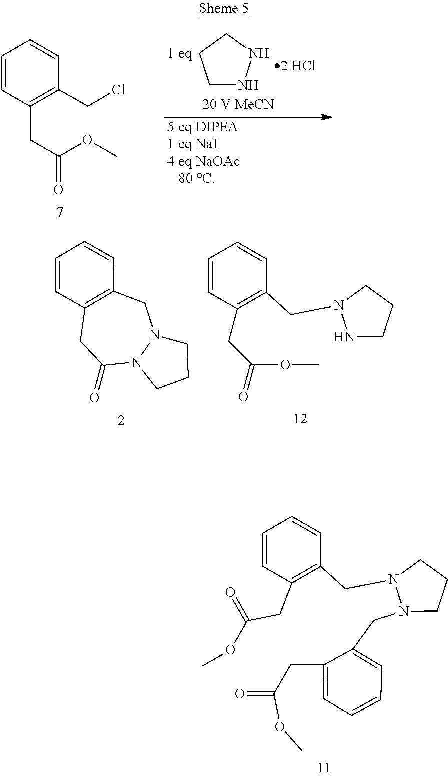

TABLE 4

HPLC area percent (%)

| Reaction time (h) | 12 | 2 | 11 | 7 |
|---|---|---|---|---|
| 0 | 30.7 | 46.6 | 14.4 | 0 |
| 1 | 14.7 | 61.9 | 14.1 | 0 |
| 2 | 6.9 | 69.2 | 14.3 | 0 |
| 4 | 1.7 | 74.1 | 14.5 | 0 |
| 6 | 0.3 | 75.2 | 14.2 | 0 |

At 0 h and 80° C., all starting material 7 was consumed and immediately converted to monomer 12, dimer 11, and product 2. As the reaction proceeded, almost all of monomer 12 was slowly converted to Compound 2 but byproduct 11 remained unchanged from 0 h to 6 h.

The reaction conditions and equivalents of pyrazolidine 10 were then investigated in an attempt to reduce the amount of byproduct 11 produced (Scheme 6, Table 5).

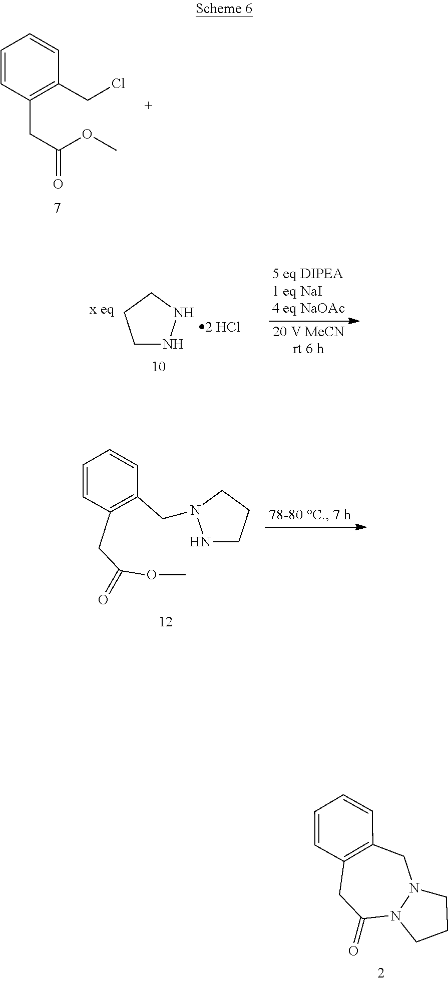 [SMILES: COC(=O)Cc1ccccc1CCl ; C1CNNC1 ; COC(=O)Cc1ccccc1CN1CCCN1 ; O=C1Cc2ccccc2CN2CCCN12]

TABLE 5

| | Pyrazolidine | Temp | Time | HPLC area percent (%) | | | |
|---|---|---|---|---|---|---|---|
| Entry | (eq) | (° C.) | (h) | 12 | 2 | 11 | 7 |
| 1 | 1 | 80 | 7 | 0 | 75.4 | 16.0 | 0 |
| 2 | 1 | rt | 6 | 66.4 | 7.9 | 11.2 | 0 |
| | | 80 | 7 | 0 | 84.5 | 11.4 | 0 |
| 3 | 1.5 | rt | 0 | 22.5 | 0 | 0.47 | 74.7 |
| | | | 5 | 79.7 | 5.1 | 4.5 | 0 |
| | | 80 | 7 | 0 | 94.0 | 4.6 | 0 |
| 4 | 2 | rt | 0 | 17.1 | 0 | 2.9 | 81.0 |
| | | | 5 | 83.0 | 4.8 | 3.0 | 0.2 |
| | | 80 | 7 | 0 | 95.3 | 2.9 | 0 |

Lowering the reaction temperature from 80° C. to room temperature reduced byproduct 11 from 16% to 11% (entries 1 and 2). Excess pyrazolidine 10 at room temperature further reduced byproduct 11 to 4.5% (1.5 eq 10) and 3% (2 eq 10) (entries 3 and 4) while 12 was increased to 80% and 83%, respectively. The intracyclization between the amine and ester group of 12 was completed at an elevated reaction temperature (80° C.) for 7 h to provide Compound 2 with about 95% yield. This two-step modification in Table 5 has been demonstrated on a hundred-gram scale producing Compound 2 with an excellent purity (99.10% by HPLC) and a higher yield (82%).

As such, Compound 2 can be prepared at high yield and excellent purity by the following procedure.

To a mixture of 7 (50 g, 249 mmol) in a degassed CH₃CN (1000 mL) was added anhydrous sodium iodide (37.4 g, 249 mmol) and pyrazolidine dihydrochloride 10 (56.8 g, 374 mmol) followed by dropwise addition of N,N-diisopropylethylamine (217 ml, 1246 mmol) below 26° C. in ~3 h and then sodium acetate (82 g, 997 mmol). The mixture was stirred at room temperature for 5 h and was further heated to 80° C. for 6 h. After cooling to rt, the mixture was filtered, concentrated and extracted with EtOAc (500 mL). The organic extraction was washed with saturated NaHCO₃ (50 mL) and brine (50 mL), dried over Na₂SO₄, and concentrated. The resulting crude residue was purified by flash chromatography (SiO₂, EtOAc/hexanes) to afford the desired product (43.2 g, 85.7% yield) as an off-white solid. LC-MS calculated for C₁₂H₁₄N₂O (M+H)⁺: m/z=203.1; found 203.1. ¹H NMR (400 MHz, DMSO-d₆) δ ppm 7.18-7.22 (m, 1H), 7.12-7.16 (m, 2H), 7.04 (d, J=5.8 Hz 1H), 4.16 (s, 2H), 3.85 (br s, 2H), 3.49 (t, J=5.5 Hz, 2H), 3.20 (t, J=5.5 Hz, 2H), 2.16-2.21 (m, 2H).

Various modifications of the disclosure, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A process for preparing Compound 1: 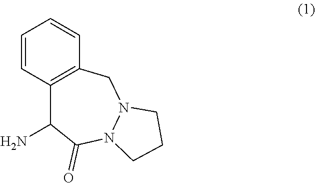 [SMILES: NC1C(=O)N2CCCN2Cc2ccccc21]
or a salt thereof;
wherein the process comprises the step of reacting Compound 2: 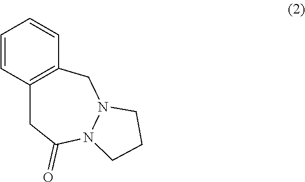 [SMILES: O=C1Cc2ccccc2CN2CCCN12]
or a salt thereof;
with an aminating agent and at least one base to produce Compound 1.

2. The process of claim 1, wherein Compound 2 is reacted with one base or two bases.

3. (canceled)

4. The process of claim 1, wherein the base is a non-nucleophilic base or a metal alkoxide.

5. The process of claim 1, wherein the base is selected from potassium bis(trimethylsilyl)amide (KHMDS), sodium bis(trimethylsilyl)amide (NaHMDS), lithium bis(trimethylsilyl)amide (LiHMDS), lithium diisopropylamide (LDA), sodium hydride (NaH), potassium hydride (KH), 1,8-diazabicyclo[5.4.0]undec-7-ene (DBU), N,N-diisopropylethylamine (DIPEA), tert-butoxide (KOtBu), sodium tert-butoxide (NaOtBu), sodium ethoxide (NaOEt), sodium methoxide (NaOMe), and potassium methoxide (KOMe).

6-10. (canceled)

11. The process of claim 1, wherein the aminating agent is selected from O-diphenylphosphinyl hydroxylamine, monochloramine, O-(trimethylsilyl)hydroxylamine, hydroxyamine-O-sulfonic acid, O-tritylhydroxylamine, (2,4-dinitrophenyl)hydroxylamine, O-mesitylenesulfonylhydroxylamine, O-(2,4,6-triisopropylbenzenesulfonyl)hydroxylamine, O-(2,4,6-trinitrophenyl)hydroxylamine, O-(2,3,4,5,6-pentafluorobenzyl)hydroxylamine, O-(2,4,6-trimethylbenzoyl)hydroxylamine, O-(4 nitrobenzoyl)hydroxylamine, O-(2,4,6-trimethylbenzyl)hydroxylamine, and O-(4-nitrophenyl)hydroxylamine.

12-16. (canceled)

17. The process of claim 1, wherein the process occurs at a temperature from about −20° C. to about −35° C.

18-20. (canceled)

21. The process of claim 1, wherein the process comprises an acid-base extraction.

22. The process of claim 21, wherein the acid-base extraction comprises the following step of treating Compound 1 with an acid to generate a salt of Compound 1.

23. (canceled)

24. The process of claim 22, wherein the acid is selected from hydrochloric acid (HCl), nitric acid (HNO₃), sulfuric acid (H₂SO₄), hydrobromic acid (HBr), perchloric acid (HClO₄), and trifluoroacetic acid (CF₃CO₂H).

25. (canceled)

26. The process of claim 22, wherein the salt of Compound 1 is washed with a polar, aprotic solvent.

27-28. (canceled)

29. The process of claim 22, wherein the acid-base extraction comprises the following step of treating the salt of Compound 1 with a base to generate Compound 1 as a free base.

30. (canceled)

31. The process of claim 29, wherein the base is selected from sodium hydroxide (NaOH), potassium hydroxide (KOH), lithium hydroxide (LiOH), potassium tert-butoxide (KOtBu), sodium tert-butoxide (NaOtBu), sodium ethoxide (NaOEt), sodium methoxide (NaOMe), and potassium methoxide (KOMe).

32. (canceled)

33. The process of claim 29, wherein Compound 1 is washed with a polar, aprotic solvent.

34-37. (canceled)

38. The process of claim 1, wherein Compound 1 is prepared at an overall yield of about 80% or greater and an overall purity of greater than 99%.

39. (canceled)

40. The process of claim 1, wherein Compound 1 is prepared without a chromatography step.

41. A process for preparing Compound 2: 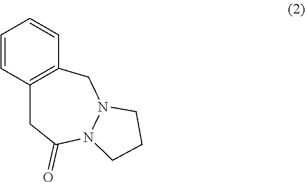 [SMILES: O=C1Cc2ccccc2CN2CCCN12]
wherein the process comprises the step of reacting Compound 3: 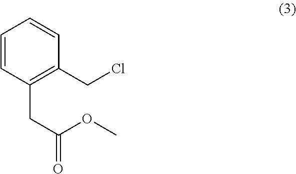
or a salt thereof;
with Compound 4: 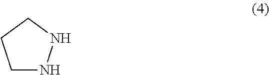
or a salt thereof;
in the presence of at least one base, a halogenating agent, and a solvent that is acetonitrile or an alcohol, to produce Compound 2.

42. The process of claim 41, wherein the solvent is acetonitrile, ethanol, or methanol.

43-44. (canceled)

45. The process of claim 41, wherein the process occurs in the presence of two bases.

46. The process of claim 41, wherein the base is a non-nucleophilic base and an alkali metal acetate salt.

47. The process of claim 41, wherein the base is selected from potassium bis(trimethylsilyl)amide, sodium bis(trimethylsilyl)amide, lithium diisopropylamide, sodium hydride, potassium hydride, 1,8-diazabicyclo[5.4.0]undec-7-ene, N,N-diisopropylethylamine, sodium acetate, and potassium acetate.

48-51. (canceled)

52. The process of claim 41, wherein the halogenating agent is an iodinating agent.

53. The process of claim 41, wherein the halogenating agent is selected from sodium iodide, N-lodosuccinimide, iodine chloride, 1,3-diiodo-5,5-dimethylhydantoin, N-iodosaccharin, and bis(pyridine)iodonium tetrafluoroborate.

54-56. (canceled)

57. The process of claim 41, wherein the process occurs at a room temperature.

58. The process of claim 57, wherein the reaction is heated to about 70° C. to about 80° C. after reacting at room temperature.

59. The process of claim 41, wherein Compound 2 is prepared at an overall yield of about 85% or greater and an overall purity of greater than 98%.

60. (canceled)

61. The process of claim 41, wherein the ratio of Compound 2 produced to byproduct produced is about 4:1, about 4.5:1, or about 5:1.

62-63. (canceled)

64. The process of claim 61, wherein the byproduct is Compound 5: 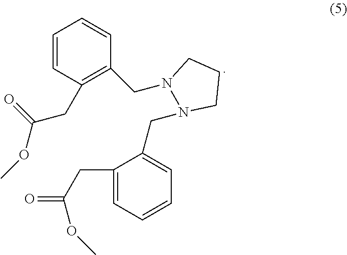 [SMILES: COC(=O)Cc1ccccc1CN1CCCN1Cc1ccccc1CC(=O)OC]
